(19) United States
(12) Patent Application Publication
(10) Pub. No.: US 2012/0094882 A1
(43) Pub. Date: Apr. 19, 2012

(54) Denture Cleanser

(76) Inventors: Leonard Mackles, New York, NY (US); William Bess, Edison, NJ (US)

(21) Appl. No.: 12/905,803

(22) Filed: Oct. 15, 2010

(51) Int. Cl. A61K 8/25; A61Q 11/00

(52) U.S. Cl. 510/116

(57) ABSTRACT

There is provided a tasteless denture cleaning composition comprising inter alia, a source of hypochlorite, a source of sulfite and precipitated silica gel.

### FIELD OF THE INVENTION

Tasteless denture cleansers

### BACKGROUND OF THE INVENTION

Denture cleanser products in tablet and powder form are well known in the art. The purpose of a denture cleanser is to remove stains, plaque, microbial contamination, etc. from the denture. A well known method is to place the denture in about 8 oz of warm water, add the tablet or powder, and allow the denture to soak until clean. The cleansing is achieved by release of oxygen from the cleanser formulation, usually by formation of hydrogen peroxide which then releases the oxygen.

More effective cleansers have been formulated using hypochlorite either added as a powder directly to the formulation or by formation during the tablet or powder solubilization in the water during soaking. The preferred method of utilizing hypochlorite is to provide chloride in the presence of a pharmacologically acceptable source of an agent capable of oxidizing chloride ion to hypochlorite, preferably using potassium monopersulfate and sodium chloride. Upon addition to the water, hypochlorite is formed by a reaction between these compounds. While the hypochlorite provides improved cleansing, there is at least one drawback to using hypochlorite in that it imparts a typical “chlorine” odor to the solution, which many consumers find objectionable.

An early patent in this field is U.S. Pat. No. 3,337,466. The use of sodium sulfite as a hypochlorite deodorizer is well known in the prior art. U.S. Pat. No. 4,295,985 discloses the use of sodium sulfite as a chlorine odor remover from skin and hair of bathers. U.S. Pat. Nos. 4,552,679 and 4,671,972 both disclose the use of sodium sulfite as a hypochlorite deodorizer in denture cleanser products, although the use of sulfites is not acceptable for use by consumers because “ The sulfur compounds replace the chlorine odor with their own sulfurous odor . . . .” Furthermore, this prior art requires the use of an encapsulated deodorizer in order to effectively reduce the hypochlorite odor.

### SUMMARY OF THE INVENTION

The improvement set forth by the present invention comprises the elimination of the sulfurous odor previously encountered when utilizing sodium sulfite as a hypochlorite deodorizer in denture cleansers, either with or without encapsulation, by the incorporation of precipitated silica gel into the composition

### DETAILED DESCRIPTION OF THE INVENTION

This invention deals with the deodorization of denture cleansers that utilize a hypochlorite generating agent when they are added to water. Specifically this invention utilizes a sulfite in combination with a precipitated silica gel as described in the US Pharmacopoeia XXII to both deodorize the hypochlorite odor, and prevent the sulfurous odor previously described in the prior art. As used herein the term odor includes the term taste.

As used in the specification and claims, the term “hypochlorite generating agent” means any compound which upon contact with water generates or releases hypochlorite ion directly or by the interaction of two or more compounds. Illustrative, non-limiting examples of the hypochlorite generating agent of this invention are heterocyclic N-chloroimides such as a chloroisocyanurate, e.g., sodium dichloroisocyanurate, potassium dichloroisocyanurate, or trichlorocyanuric acid; complex salts of two or more compounds, e.g., [(mono-trichloro)-tetra(monopotassiumdichloro)]penta-isocyanurate; other N-chloroimides, e.g., sodium-p-toluene-sulfonochloramide, N,N-dichloro-p-toluenesulfonamide, sodium benzene-sulfonchloramide, N,N-dichlorobenzene-sulfonamide, N-chlorosuccinate. Still other compounds which liberate hypochlorite/chlorine on contact with water are N-chloro malonimide, N-chloro phthalinide and N-chloro naphthalimide; the hydantoins such as 1,3-dichloro-5,5-dimethyl hydrantoin; N-monochloro-C,C-dimethylhycrantoin; methylene-bis-(N-chloro-C,C-dimethylhydantoin); 1,3-dichloro-5-ethylhydantoin and 1,3-dichloro-5,5-diisoethylhydantoin; dry solid inorganic compound such as lithium hypochlorite, calcium hypochlorite; mixtures of salts, such as sodium persulfate and sodium chloride; sodium perborate and chlorinated 5-triazine triones.

The sulfite cation may be an alkali metal cation, suitably sodium or potassium and the source of sulfite may be selected from the group consisting of sulfites, bisulfites, metabisulfites, and hydrosulfites

The amounts of sulfite and precipitated silica gel in the dry composition (by weight of the entire composition) range between from about 0.05% to 5%, preferably from about 0.1% to 0.5%, for each of these components

It will be recognized that the immediate invention is not limited by the following examples, and that additional formulation ingredients known in the art, for example flavors, and colorants, can be incorporated or substituted for ingredients in the examples. The suitable formulations of the present invention are set forth below

### EXAMPLE 1

| Ingredient | % by Weight |
|---|---|
| Potassium Monopersulfate Anhydrous | 30.00 |
| Sodium Perborate, Monohydrate | 10.00 |
| Sodium Tripolyphosphate | 20.00 |
| Sodium Sesquicarbonate | 28.80 |
| Tetra Sodium EDTA | 0.50 |
| Sodium Lauryl Sulfate | 0.50 |
| Sodium Metasilicate, Anhydrous | 5.00 |
| Sodium Chloride | 5.00 |
| Precipitated Silica Gel | 0.10 |
| Sodium Sulfite | 0.10 |
| | 100.00 |

### EXAMPLE 2

| Ingredient | % by Weight |
|---|---|
| Potassium Monopersulfate Anhydrous | 25.00 |
| Sodium Perborate, Monohydrate | 15.00 |
| Sodium Tripolyphosphate | 20.00 |
| Sodium Sesquicarbonate | 33.00 |
| Tetra Sodium EDTA | 0.50 |
| Sodium Lauryl Sulfate | 0.50 |
| Sodium Chloride | 5.00 |
| Precipitated Silica Gel | 0.50 |
| Sodium Sulfite | 0.50 |
| | 100.00 |

### EXAMPLE 3

Taste Test

A panel of 5 subjects were asked to smell the solution formed by adding 2.5 grams of the formulation in Example 2 to 200 ml of warm water. None of the 5 subjects smelled any chlorine, hypochlorite, or sulfurous odor. The same 5 subjects were asked to smell a similar solution as Example 2, but without the precipitated silica gel. None of the 5 subjects smelled any chlorine or hypochlorite odor, but 4 of the 5 reported a slight sulfurous odor.

### EXAMPLE 4

| Ingredient | % by Weight |
|---|---|
| Potassium Monopersulfate Anhydrous | 30.000 |
| Sodium Perborate, Monohydrate | 10.000 |
| Sodium Tripolyphosphate | 20.000 |
| Sodium Sesquicarbonate | 29.995 |
| Tetra Sodium EDTA | 0.500 |
| Sodium Lauryl Sulfate | 0.500 |
| Sodium Metasilicate, Anhydrous | 2.500 |
| FD&C Blue #1 | 0.005 |
| Peppermint Flavor | 0.250 |
| Sodium Chloride | 5.000 |
| Precipitaed Silica Gel | 1.000 |
| Sodium Sulfite | 0.250 |
| | 100.000 |

In the above examples, the product is intended to be dispensed as a free flowing powder. Tablets can be prepared from any of the examples by the addition of 0.2% magnesium stearate and removing 0.2% of sodium sesquicarbonate.

1. In a denture cleanser comprising a water soluble cleansing agent for cleansing a denture immersed in a solution thereof, comprising a member selected from the group consisting of hypochlorite and a hypochlorite generating agent which releases hypochlorite in the presence of a pharmacologically acceptable source of an agent capable of oxidizing said hypochlorite generating agent to hypochlorite, and a source of sulfite, the improvement comprising further providing precipitated silica gel sufficient to remove both sulfurous and chlorine odor from said [a] solution [of said components].

2. The composition of claim 1 where the sulfite is selected from the group consisting of sulfites, bisulfites, metabisulfites, and hydrosulfites.

3. The composition of claim 2 where the sulfite is an alkali metal sulfite.

4. The composition of claim 3 where the alkali metal is sodium, or potassium.

5. The composition of claim 1 where the sulfite content is from about 0.05% to 5% by weight of the total composition.

6. The composition of claim 1 where the sulfite content is from about 0.1% to 0.5% by weight of the total composition.

7. The composition of claim 1 where the precipitated silica gel content is from about 0.05% to 5% by weight of the total composition.

8. The composition of claim 7 where the precipitated silica gel content is from about 0.1% to0.5% by weight of the total composition.

9. A method for cleaning dentures by adding the composition of claim 1 to water, and soaking the dentures in the resulting combination.
